(19) United States
(12) Patent Application Publication
(10) Pub. No.: US 2009/0268864 A1
(43) Pub. Date: Oct. 29, 2009

(54) BREAST IMAGE GENERATING METHOD

(75) Inventor: Naoto Nishida, Fukuoka (JP)

Correspondence Address: FRISHAUF, HOLTZ, GOODMAN & CHICK, PC, 220 Fifth Avenue, 16TH Floor, NEW YORK, NY, 10001-7708, US

(73) Assignee: KONICA MINOLITA MEDICAL & GRAPHIC INC., TOKYO (JP)

(21) Appl. No.: 11/915,988

(22) PCT Filed: Apr. 14, 2006
(86) PCT No.: PCT/JP2006/308512

§ 371 Date: Nov. 29, 2007

(30) Foreign Application Priority Data
Jun. 2, 2005 (JP) 2005-162455

(51) Int. Cl. A61B 6/04; H05G 1/64

(52) U.S. Cl. 378/37; 378/98.12

(57) ABSTRACT

A breast image generating method correlates radiographing order information for instructing to radiograph each of left and right breasts of a subject in an oblique direction with identification information indicating a relationship between data of a breast image and the radiographing order information so as to be stored in a storing device. A breast is radiographed based on the radiographing order information to generate data of a breast image, radiographing order information corresponding to the generated data of a breast image is identified based on the identification information, and a stamp image is superposed at a prescribed position on the breast image taken through radiographing conducted in the oblique direction to make a composite image in accordance with whether a breast region indicated by the identified radiographing order information is left or right, so as to display the composite image on a displaying device.

### TECHNICAL FIELD

The present invention relates to a method to generate breast images for a left breast and a right breast of one patient.

### BACKGROUND OF THE INVENTION

There has been developed a radiographing system wherein an X-ray radiographing apparatus for radiographing a breast region for inspection of a breast cancer or the like and its control device are connected to each other through a communication network to simplify control work for radiographing (for example, see Patent Document 1).

In the aforesaid radiographing system, an instruction information concerning radiographing such as radiographing order information is issued by the request of an doctor before radiographing, and radiographing jobs are advanced by a radiographing technician based on the radiographing order information. The radiographing order information is an instruction information including information of a patient such as a name and gender of a patient, information about radiographing such as a radiographing region, a radiographing direction and a radiographing method, and information about inspection such as a date of inspection and a name of a doctor in charge. After breast images are generated through radiographing conducted based on the radiographing order information, the breast images are accompanied with the radiographing order information in the radiographing system, as information concerning breast images. After that, the breast images are converted into data base based on that accompanying information, to be controlled.

When generating breast images, plural radiographing directions are used for radiographing for left and right breasts of one patient, in many cases. Therefore, a plurality of cassettes are sometimes used for continuous radiographing. When using plural cassettes, if the relationship between the radiographing order information and the cassette used becomes unclear, it becomes impossible to recognize which radiographing order information should be accompanied with breast image read out of the cassette in the radiographing system. Therefore, processing for registration for setting to cause radiographing order information to correspond to unique ID of cassette (which is called cassette registration) before or after radiographing is necessary. Patent Document 1: Japanese Patent Publication Open to Public Inspection No. 2002-158862

### DISCLOSURE OF THE INVENTION

### Problems to be Solved by the Invention

When the cassette registration is conducted before radiographing, relationship of correspondence between radiographing order information for an object to be radiographed and the registered cassette needs to be remembered by a technician, because the technician is requested to radiograph by using the cassette registered for the radiographing order information. On the other hand, when the cassette registration is conducted after radiographing, the relationship between the radiographing and the cassette used during the radiographing also needs to be remembered. When a technician makes a mistake for the relationship between the cassette and the radiographing, respective breast images obtained through a series of radiographing are accompanied with wrong radiographing order information, in a control device.

The present invention has been achieved in view of the problems stated above, and its objective is to simplify telling right from wrong for the relationship of correspondence between breast images and radiographing order information necessary for control of the images, and to allow right correction for an error.

### Means to Solve the Problems

Objectives of the invention mentioned above are achieved by the following structures.

(1) A breast image generating method characterized by including a process to store in a storing device the radiographing order information that instructs radiographing in the oblique directions for left and right breasts of a subject and identification information showing the relationship between data of breast image and the radiographing order information, by making them to correspond to each other, a process to generate data of breast images by radiographing breasts based on the radiographing order information, a process to identify radiographing order information corresponding to data of the aforesaid generated breast images based on the aforesaid identification information and a process to superpose stamp images at a prescribed position on the breast image taken in the oblique direction depending on whether the breast region indicated by the aforesaid identified radiographing order information is left or right after the radiographing order information is identified concerning data of breast images equivalent to one subject, and to display it on the display device.

(2) A breast image generating method characterized by including a process to store in a storing device the radiographing order information that instructs radiographing in the oblique directions and vertical directions for left and right breasts of a subject and identification information showing the relationship with data of breast image and the radiographing order information, by making them to correspond to each other, a process to generate data of breast images by radiographing breasts based on the radiographing order information, a process to identify radiographing order information corresponding to data of the aforesaid breast images based on the aforesaid identification information and a process to superpose stamp images at prescribed position on the breast image taken in the oblique direction depending on whether the breast region indicated by the aforesaid identified radiographing order information is left or right after the radiographing order information is identified concerning data of breast images equivalent to one subject, and to display it on the display device.

(3) The breast image generating method according to Item (1) or Item (2) wherein, when correction of the aforesaid identification information is instructed and operated through an operation device after the composite image on which the stamp images are superposed is displayed, respective breast images equivalent in terms of amount to one subject are arranged and displayed side by side in the order of generating the breast images.

(4) The breast image generating method according to Item (3) wherein, there is included a process to correct identification information corresponding to the radiographing order information in accordance with instruction and operation of correction of correspondence relationship between the breast images and radiographing order information inputted through an operation device, after respective breast images are displayed in the order of generating images, and to cause the storing device to update and to store.

(5) The breast image generating method according to Item (4) wherein, there is included a process to identify radiographing order information corresponding to each of breast images of one set for the left and right taken in the oblique directions, based on the corrected identification information, and a process to superpose stamp images at prescribed position on breast image taken in the oblique direction for composition in accordance with whether a breast region indicated by the identified radiographing order information is left or right, and to display it on the display device again.

### EFFECTS OF THE INVENTION

In Items (1) and (2), it is easy for a technician to discriminate the correct and the incorrect of the correspondence relationship between breast images for left and right breasts taken in the oblique directions and radiographing order information, based on the positional relationship between stamp images based on radiographing performance conditions and radiographing order information and the breast area in breast images.

In Item (3), it is possible to determine whether a breast region of each breast image is left or right, from the order of generating breast images. Since radiographing is conducted alternately for left and right breasts, in general, when breast images are arranged in the order of generation of them, breast regions of each breast image should be alternative for left and right. Further, since it is possible to distinguish left and right breasts visually from the shape of the breast area in images, for breast images in the oblique direction, the breast region of breast image in the vertical directions can be distinguished based on the order of generation of respective breast images, by identifying the breast image in the oblique direction.

In Item (4), it is possible to correct the correspondence relationship between radiographing order information and breast images by correcting identification information that is a key to cause radiographing order information and breast images to correspond to each other.

In Item (5), it is possible for a technician to comfirm whether the correct correspondence relationship between radiographing order and breast images has been achieved or not due to the correction operation by displaying composite images where stamp images are superposed again by the correspondence relationship after correction.

### BRIEF DESCRIPTION OF THE DRAWINGS

FIG. 1 is a diagram showing the system structure of breast image radiographing system 100 in the present embodiment.

FIG. 2 is a diagram showing a radiographing apparatus shown in FIG. 1.

FIG. 3 is a diagram illustrating an example of loading a cassette on a radiographic stand.

FIG. 4 is a diagram showing an internal structure of a control device.

FIG. 5 is a diagram showing an example of an order file for controlling correspondence relationship for radiographing order information, cassette ID and radiographing performance conditions.

FIG. 6 is a flow chart illustrating image registration processing operated in the control device.

FIG. 7 is a diagram showing an example of preparing composite images in the case where radiographing order information and breast images are in a correct correspondence relationship.

FIG. 8 is a diagram showing an example of preparing composite images in the case where radiographing order information and breast images are in a wrong correspondence relationship.

Each of FIGS. 9(a) and 9(b) is a diagram showing an example of a display screen page displayed in the control device, and FIG. 9 (a) shows a confirmation screen page, while, FIG. 9 (b) shows a screen page for results of correction.

FIG. 10 is a diagram showing an example of a correction screen page displayed in the control device.

FIG. 11 is a diagram showing a confirmation screen page in the case of radiographing in different radiographing directions.

FIG. 12 is a diagram showing a correction screen page in the case of radiographing in different radiographing directions.

### EXPLANATIONS OF SYMBOLS

100. Breast image radiographing system
10. Radiographing apparatus
30. Control device
31. Control section
32. Operation section
33. Display section
36. Image memory
50. Reading device
c. Cassette

### PREFERRED EMBODIMENT OF THE INVENTION

First, the structure will be described as follows.

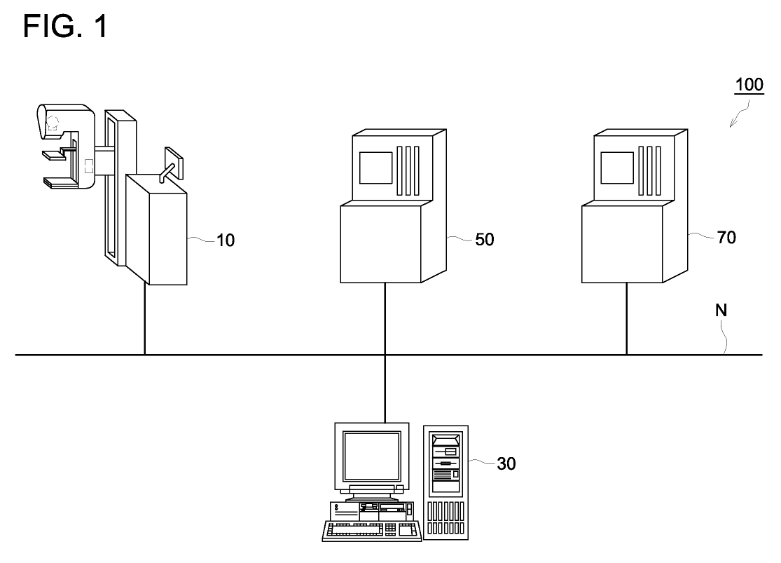

FIG. 1 shows the system structure of breast image radiographing system 100 in the present embodiment.

The breast image radiographing system 100 is one wherein a breast of a patient (subject) is radiographed as a radiographing region, and data of that breast image are generated, and image control is carried out by causing the image to be accompanied by radiographing order information. As shown in FIG. 1, the breast image radiographing system 100 is composed of radiographing apparatus 10, control device 30, reading device 50 and film outputting device 70, and respective devices and apparatus are connected each other through communication network N.

Each of the aforesaid items will be described as follows.

The radiographing apparatus 10 is one that radiographs a breast of a patient as a subject, and records the radiograph (breast image) on a cassette.

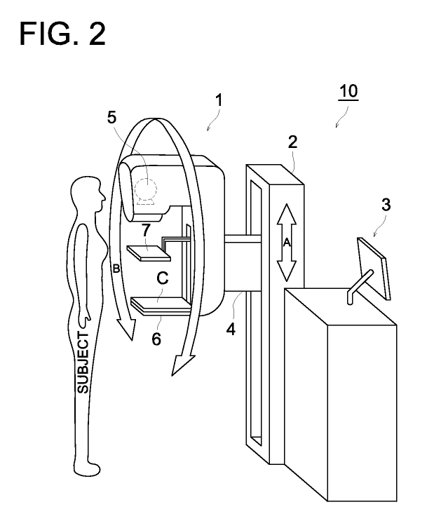

FIG. 2 shows radiographing apparatus 10.

As shown in FIG. 2, radiographing section 1 of the radiographing apparatus 10 is formed to be in an arm shape, and is made up so that it may go up and down in direction A along columnar support 2, and may rotate in direction B through support shaft 4. Further, the radiographing apparatus 10 has main body section 3 for conducting operations of the radiographing section 1 to go up and down and to rotate and operations of communication with control device 30, and a technician can radiograph through this main body section 3.

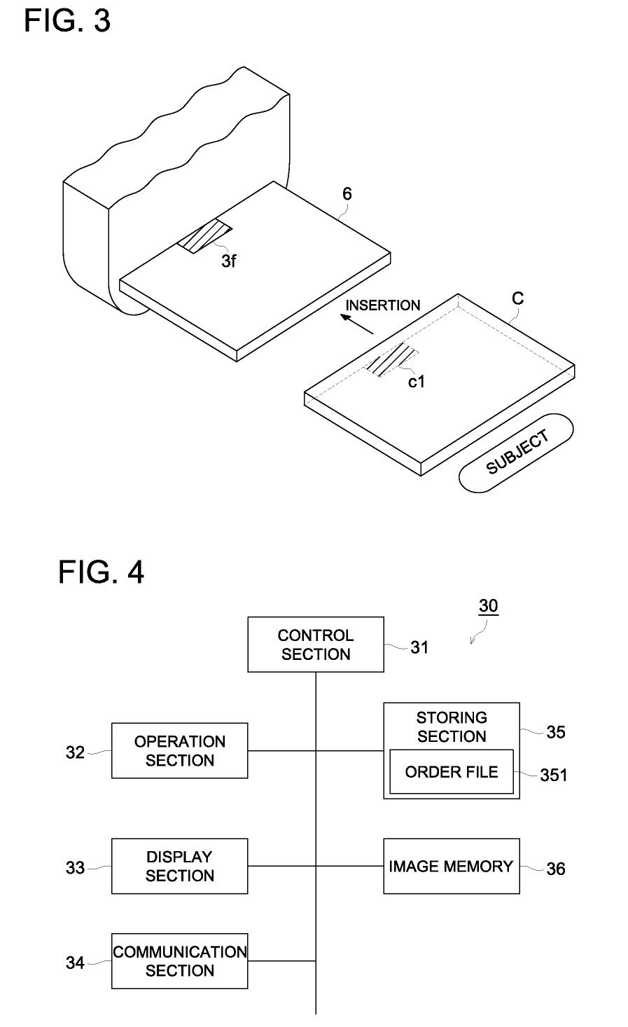

In the radiographing section 1, X-ray source 5 that emits X-rays and radiographic stand 6 for loading a breast are arranged to face each other, and the radiographing section 1 is composed of pressure plate 7 that nips and presses a breast placed on the radiographic stand 6. FIG. 3 shows a side view of the radiographing section 1. As shown in FIG. 3, the radiographing section 1 is constructed so that an image detector (cassette c) may be loaded on the upper part of the radiographic stand 6, and a holder (not shown) for fixing the cassette c on the radiographic stand 6 is formed on the radiographing section 1. Though an example in which the cassette c is applied as an image detector is described in the present embodiment, in this connection, other image detectors such as FPD (Flat Panel Detector) and others may also be applied.

The radiographing direction can be adjusted by rotating the radiographing section 1 in direction arrow B shown in FIG. 2. For example, when radiographing in the vertical direction (hereinafter, referred to as CC), the radiographing section 1 is caused to stand upright (rotation angle is 0°) for radiographing, while, when radiographing in the oblique direction (hereinafter, referred to as MLO), the radiographing section 1 is rotated by about 30° from the upright state for radiographing. In this connection, radiographing in MLO in the invention means radiographing in the rotation angle (for example, 15° or 60°) excluding 0° in the case of radiographing in CC, without being limited to radiographing conducted by rotating the radiographing section 1 by 30°. On the breast image taken through radiographing in MLO, there exists a pectoral muscle section, and a position for this pectoral muscle section is always different between left and right breast images, thus, a technician can distinguish left and right of breast regions easily and visually based on a position of this pectoral muscle section, as stated later.

The cassette c is one having therein a built-in phosphor plate to record thereon radiographs by absorbing X-rays transmitted through a subject. Incidentally, the cassette c includes one of a common type usable also for radiographing regions other than breasts and one used exclusively for breasts. Although any type of cassette c can be used, it is preferable to color, to provide irregularities or to change materials so that a technician can discriminate one side to be positioned on the subject side of the cassette ct for the purpose of unifying arrangement directions for cassette c on radiographic stand 6. Incidentally, in the case of the cassette used exclusively for breasts, the phosphor plate is formed so that its one side can be used up to a margin of a chest wall of a subject, and it is general that the aforesaid processing is carried out so that one side that is brought into contact with a subject can be discriminated.

On the cassette c, there is provided cassette ID (identification information in the invention) as identification information unique to the cassette generally, and this cassette ID is shown as a bar code, to be displayed on the other side opposite to one side that comes in contact with a subject in cassette c as shown in FIG. 3. Corresponding to this, there is provided, on radiographic stand 6, ID reading section 3f that reads a bar code at the position corresponding to the portion (which is called ID section c1) where its bar code is displayed when the cassette c is arranged When ID section c1 of cassette c loaded on radiographic stand 6 was read correctly by this ID reading section 3f, the cassette c is judged to be loaded in the correct arrangement direction, while, when ID section c1 was not read, the direction of arrangement of cassette c is judged to be wrong.

Further, after radiographing is carried out, the radiographing apparatus 10 transmits to control device 30 the information of conditions at the time of conducting radiographing such as tube current, tube voltage in X-ray source 5 during the radiographing and pressing thickness by pressure plate 7 (hereinafter referred to as radiographing performance conditions).

Next, the control device 30 will be described.

The control device 30 conducts radiographing control based on radiographing order information, and controls breast images read by reading device 50.

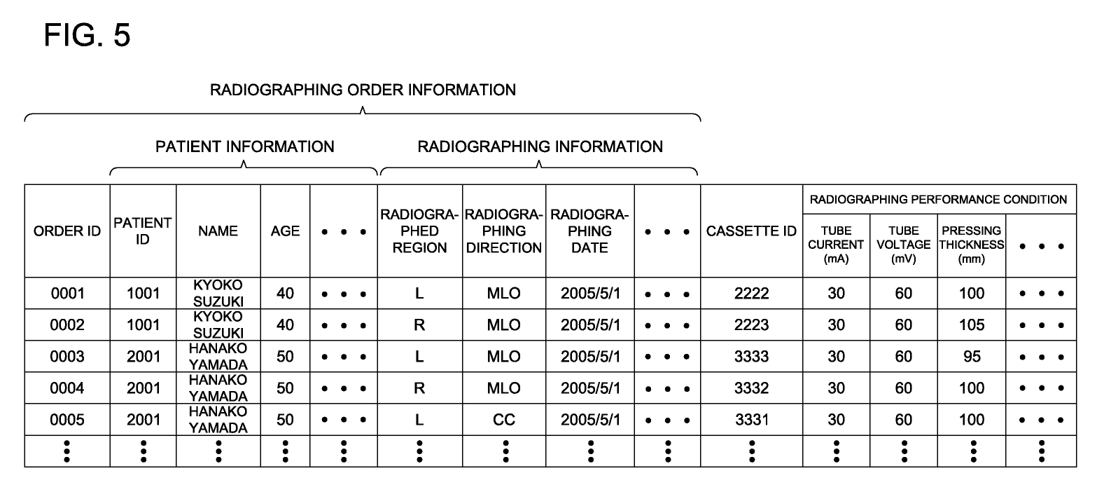

FIG. 4 shows an internal structure of the control device 30.

As shown in FIG. 4, the control device 30 is composed of control section 31, operation section 32, display section 33, communication section 34, storing section 35 and image memory 36.

The control section 31 is composed of CPU (Central Processing Unit) and RAM (Random Access Memory), and a control program stored in storing section 35 is loaded on RAM by CPU, and processing operations of each section are controlled collectively in accordance with the control program. For example, the control section 31 conducts display control for radiographing order information stored in the storing section 35, before radiographing. Further, according to registration operations inputted through operation section 32, radiographing order information is caused to correspond to the cassette ID and stored. On the other hand, when information of radiographing performance conditions at the time of radiographing is inputted from radiographing apparatus 10 through communication section 34, the information of radiographing performance conditions is stored in storing section 35 after being correlated with radiographing order information.

Further, after radiographing, the control section 35 performs image registration processing in accordance with the image registration processing relating to the invention, then, identifies radiographing order information corresponding to each breast image obtained through radiographing, and conducts processing to cause the aforesaid information to accompany the breast image. Further, the control section conducts processing for correcting its correspondence relationship. The details of the processing will be described later.

The operation section 32 represents operation devices including a key board, a mouse and a touch panel constructed solidly together with display section 33, and it generates operation signals corresponding to operations and outputs them to the control section 31.

The display section 33 is a display device that is composed of an indicating display such as LCD (Liquid Crystal Display), and it displays indicating information such as results of processing or messages, in addition to various operation screen pages such as a list screen for radiographing order information and a radiographing result screen of breast images inputted from reading device 50.

The communication section 34 is composed of interfaces for communication such as a network interface card and a modem, and it conducts receiving and transmitting data with outer equipment such as radiographing apparatus 10 and reading device 50. For example, it receives, before radiographing, radiographing order information issued in an unillustrated HIS (Hospital Information System) or RIS (Radiology Information System). Or, it receives information of radiographing performance conditions from radiographing apparatus 10, and receives data of generated breast images from reading device 50.

The storing section 35 stores a parameter necessary for the control program and for performing the control program, or stores data such as results of processing. Further, the storing section 35 is a storing device to store order file 351 for controlling correspondence relationship for radiographing order information, radiographing performance conditions and information for identifying a registered cassette.

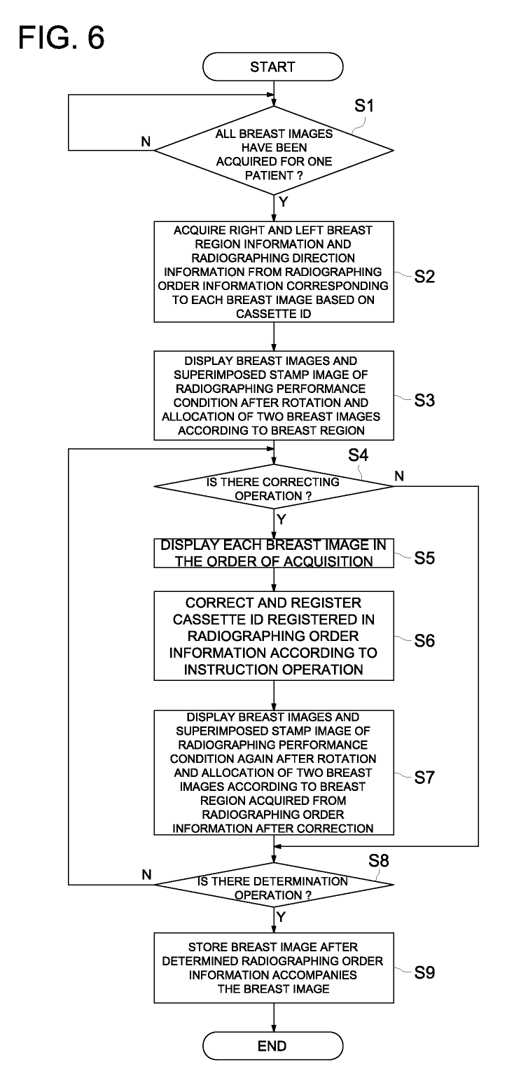

As shown in FIG. 5, there are stored, in order file 351, radiographing order information for each order ID and information of cassette ID for cassette c registered for the radiographing order information and radiographing performance conditions.

In the radiographing order information, there are included pieces of information for instructing radiographing such as patient information (patient ID, a patient name) concerning a patient to be radiographed and radiographing information (regions to be radiographed, radiographing directions) concerning radiographing. Incidentally, in the item of regions to be radiographed, “L” represents a left breast and “R” represents a right breast.

The image memory 36 stores breast images generated by reading device 50.

Next, the reading device 50 will be described.

The reading device 50 is one that conducts reading processing of breast images detected by cassette c, and generates breast image data. The reading device 50 is composed of a reading section that reads a breast image by irradiating a phosphor plate of the loaded cassette c with excitation light and by converting its stimulated light photoelectrically, a communication section for communicating with control device 30 and an operation section.

Further, the reading device 50 is equipped with a bar code reader at the position corresponding to the ID section c1 of the loaded cassette c, and this bar code reader reads cassette ID. When the cassette ID cannot be read correctly by the bar code reader, an alarm sounds, and reading processing is prohibited. This makes it possible to unify directions for loading cassette constantly.

Breast images thus read are transmitted to control device 30, after being accompanied with cassette ID information by reading device 50.

The film outputting device 70 outputs breast images on a film, based on data of breast images transmitted from control device 30.

Next, operations of the aforesaid breast image radiographing system 100 will be described, together with radiographing work of a technician.

First, a flow covering from radiographing to generation of data of breast images will be described.

In the breast image radiographing system 100, patients to be radiographed are tabulated to be displayed based on radiographing order information in control device 30 first. When a patient to be radiographed is selected by a technician from these tabulated patients, radiographing order information for the selected patient is displayed.

Incidentally, in the present embodiment, explanation will be given as follows, under the assumption that the radiographing order information instructs to radiograph in the radiographing direction of MLO for each of left and right breast regions for one patient.

A technician conducts cassette registration for two cassettes c to be used for radiographing, for each radiographing order information about left and right breast regions. To be concrete, after the radiographing order information for which cassette registration is conducted is selected by the technician, cassette ID is inputted through operation section 32. In the control device 30, information of cassette ID inputted to be corresponding to the selected radiographing order information is stored in order file 351.

Then, the technician moves to a radiographing room where the radiographing apparatus 10 is installed, while carrying two registered cassettes c, and loads cassette c that corresponds to radiographing order information to be used for radiographing on radiographic stand 6, namely, cassette c registered for the radiographing order information. In this case, the loading direction for cassette c is fixed constantly, so that ID section c1 may face ID reading section 3f on the radiographing apparatus 10 side. In the radiographing apparatus 10, reading processing for ID section c1 of the loaded cassette c is conducted by ID reading section 3f, and when the reading is incorrect, the cassette c1 is judged to be wrongly loaded, and warning actions are exhibited.

On the other hand, when the cassette c is loaded correctly, and cassette ID is read correctly, the radiographing apparatus 10 becomes ready for radiographing. Thus, the technician causes a patient representing a subject to stand at the radiographing position, and conducts instructing operations for radiographing through an operation section, after adjusting regions to be radiographed and radiographing directions, In the radiographing apparatus 10, X-ray is emitted in accordance with instructions for radiographing and radiographing is carried out. After radiographing, in the radiographing apparatus 10, pieces of information of radiographing performance conditions such as tube voltage, tube current and pressing thickness are generated, and are transmitted to control device 30 together with information of cassette ID read out by ID reading section 3f. In the control device 30, information of the radiographing performance conditions and corresponding radiographing order information are stored in order file 351 after being correlated with each other based on cassette ID.

The radiographing process of this kind is carried out, while replacing cassettes c registered for left and right breast regions.

Then, after all radiographing operations are finished, the technician loads successively two cassettes c which were finished in terms of radiographing in reading device 50. In this case, there is no restriction for the order for loading. In the reading device 50, cassette ID and breast images are read out of the loaded cassette c, and data thereof are generated.

In the control device 30, when a breast image corresponding to cassette ID registered for radiographing order information is acquired from the reading device 50, image registration processing which will be described later is performed, and the breast image is accompanied with radiographing order information.

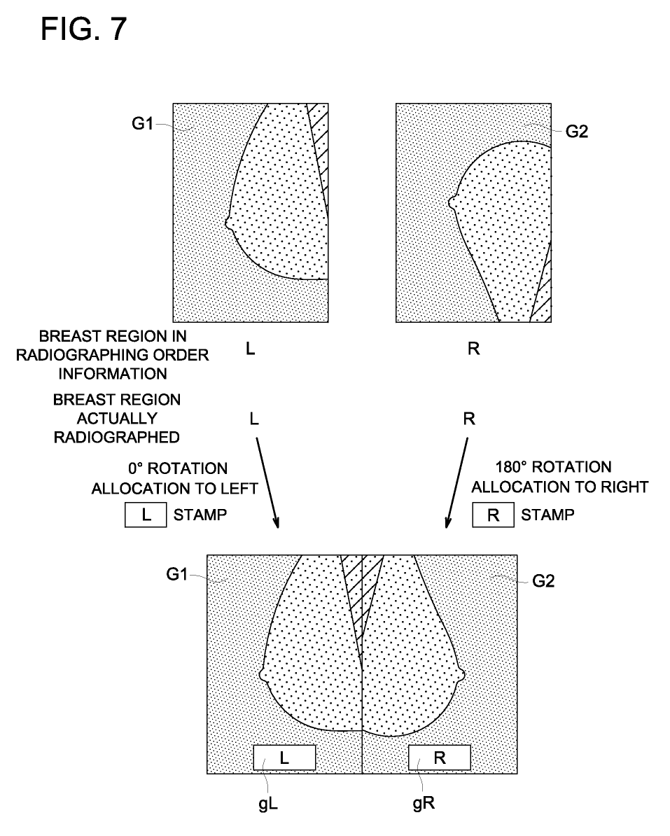

The image registration processing relating to the present embodiment will be described as follows, referring to FIG. 6.

In the image registration processing shown in FIG. 6, order file 351 is read out by control section 31 first, and it is determined whether all breast images corresponding to radiographing order information equivalent to one patient are acquired or not (step S1). This determination is made based on cassette ID, and it is determined based on whether the breast image corresponding to cassette ID registered for radiographing order information is acquired or not.

Then, after all breast images equivalent to one patient are determined to be acquired (step S1; Y), the radiographing order information corresponding to each breast image is read out of order file 351, based on cassette ID accompanying each breast image, thus, pieces of information including regions of left and right breasts, radiographing directions and radiographing performance conditions are acquired from the radiographing order information (step S2).

Then, stamp images showing radiographing performance conditions and radiographing order information concerning each breast image are prepared. Further, there is prepared a composite image wherein breast images are rotated corresponding to regions of left and right breast so that the pectoral muscle side of each breast image may adjoin the next one, and the aforesaid stamp images are superposed on the composite image, to be displayed on display section 33 (step S3).

How to prepare the composite image will be described specifically.

When interpreting breast images, it is general to interpret through comparison by arranging breast images for left and right breasts, and even when outputting to a film, according to the aforesaid reading style, a composite image is prepared to be outputted in many cases by adjusting the orientation so that the pectoral muscle side may adjoin the next one, for one set of breast images of left and right breasts which are radiographed in the same radiographing direction.

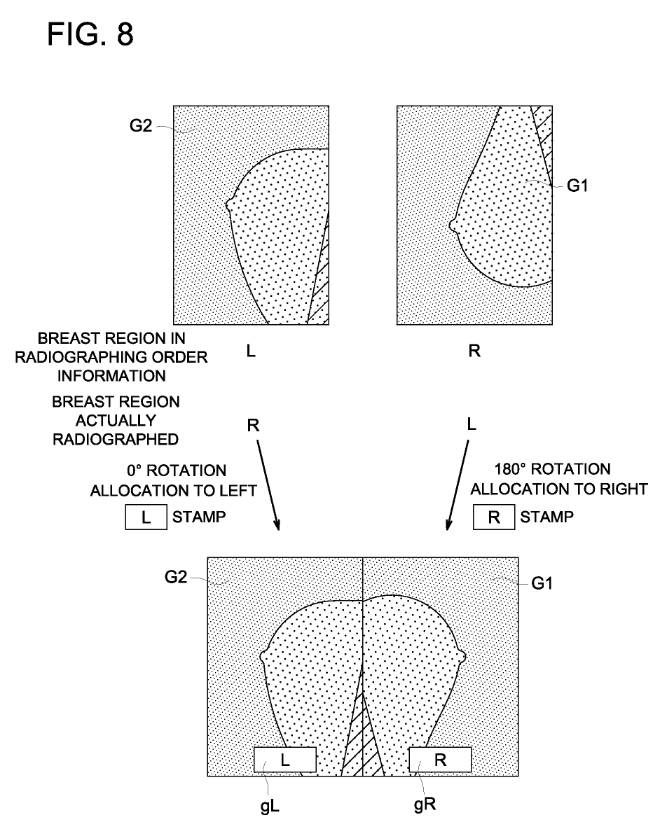

For example, when compounding breast images taken through radiographing in MLO for each of left and right breast regions, as shown in FIG. 7, breast image G1 whose radiographing direction is MLO and a breast region is a left breast (hereinafter referred to as MLO-L) is arranged to the left side position with rotation of 0°, in other words, without being rotated. On the other hand, breast image G2 whose radiographing direction is MLO and a breast region is a right breast (hereinafter referred to as MLO-R) is arranged to the right side of MLO-L after being rotated by 180°.

Incidentally, the conditions wherein a rotation angle of the breast image corresponding to the radiographing order information is 0° and its position of arrangement is on the left side if the breast region of the radiographing order information is L, while, a rotation angle of the breast image corresponding to the radiographing order information is 180° and its position of arrangement is on the right side if the breast region of the radiographing order information is R, are assumed to be stored in storing section 35 in advance as set information, and its set information is read out depending on whether a breast region of a breast image to be compounded is left or right when preparing a composite image.

When compounding of two breast images G1 and G2 terminates, stamp image gL of breast image G1 of MLO-L is prepared to be arranged at the lower and central position of breast image G1 of MLO-L to be superposed In the case of MLO-R, stamp image gR of breast image G2 of MLO-R is prepared to be arranged at the lower and central position of breast image G2 of MLO-R after compounding, to be superposed, in the same way. The stamp images gL and gR include character images showing radiographing performance conditions such as tube current and tube voltage or character images showing radiographing order such as radiographing region, and the stamp images gL and gR are outputted at a position where the stamp image is not overlapped with a breast area, namely, at a hollow area (an area irradiated directly by X-rays through no subject), thus, the stamp images are arranged at a prescribed position determined in advance as an area where a breast area does not appear on the image, such as a position that is equally distant from left and right edges of each of breast images G1 and G2, for example, and is distant from the lower part by 5 mm. Incidentally, a position where the stamp images gL and gR are arranged is established in advance and is stored storing section 35, and after preparing stamp images gL and gR based on radiographing performance condition and radiographing order information, arrangement information stored in storing section 35 is read out, and stamp images gL and gR are arranged respectively on breast images G and G2.

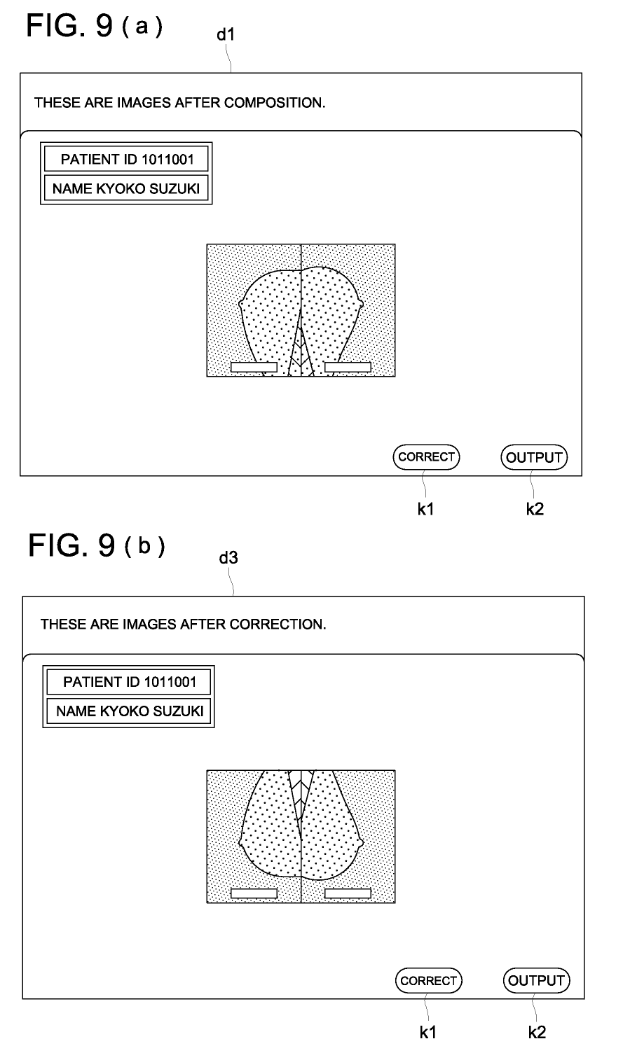

However, when a breast region designated by radiographing order information is different from a breast region which was actually radiographed, in other words, when rotation and arrangement of each breast image is conducted based on information of a breast region in the radiographing order information under the wrong correspondence relationship of a breast image, the position of the aforesaid stamp image becomes inappropriate. For example, as shown in FIG. 8, when radiographing is carried out by using a cassette on the opposite way, regular radiographing information for breast image G1 and that for breast image G2 are respectively correlated oppositely, and a breast region radiographed actually and a breast region in the radiographing order information become different from each other.

Even in this case, breast images G1 and G2 are compounded based on a breast region in radiographing order information, whereby, if compounding is carried out in accordance with arrangement conditions and rotation conditions stipulated in the aforesaid set information, the direction of arrangement of a breast area is reversed, and stamp images gL and gR are overlapped with a breast area, although the pectoral muscle area in the composite image adjoins the next one. In this case, this overlapping of the stamp images gL and gR makes it possible for a technician to determine visually the wrong correspondence relationship between radiographing order information and breast images. In the present embodiment, it is assumed that the wrong correspondence relationship can be corrected because of the positional relationship between stamp images and breast areas on this composite image.

The composite image on which stamp images are compounded is displayed on confirmation screen page d1 as shown in FIG. 9 (a). A technician confirms image quality of the composite image, presence or absence, contents and a position of a stamp image displayed on the confirmation screen page d1, and when an error of the correspondence relationship between radiographing order information and breast images due to overlapping of stamp images and breast area is found, the technician presses correction button k1. It the composite image displayed is acceptable on the other hand, output button k2 is pressed.

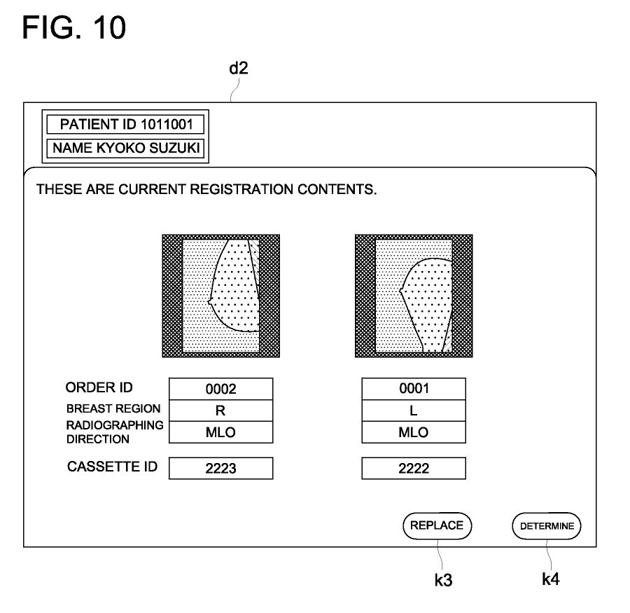

In the control device 30, when correction button k1 is pressed and correction is instructed (step S4; Y), correction screen page d2 shown in FIG. 10 is displayed on display section 33, and each breast image before compounding, that is, each breast image under the original state wherein the breast image is neither rotated nor arranged is displayed in the order of acquisition thereof (step S5). On the correction screen page d2, order ID of radiographing order information corresponding to each breast image based on cassette ID, and information of breast region and radiographing direction are displayed as contents registered presently, and cassette ID of the cassette that is registered as a cassette for the radiographing order information is displayed The technician confirms contents of the radiographing order information and breast images, and when the technician judges that the correspondence relationship between the radiographing order information and breast images is wrong, based on the position of a pectoral muscle section in two breast images, the technician presses replacement button k3.

In the control device 30, when the order replacement button k3 is pressed, cassette ID corresponding to present radiographing order information of each breast image is replaced to be updated and stored, and the correspondence relationship between each breast image and radiographing order information is corrected (step S6). In other words, the correspondence relationship is corrected by causing cassette ID corresponding to the radiographing order information to agree with cassette ID that is accompanying the real breast image corresponding to the radiographing order information.

Then, the radiographing order information corresponding to cassette ID of each breast image (namely, radiographing order information after the correspondence relationship is corrected) is read out of order file 351, and a composite image of each breast image is prepared again based on information of breast regions included in the radiographing order information. In other words, a stamp image is compounded after rotation is made depending on whether the breast region is on the left or right and further after positioning is made at the location suitable for a format for compounding. This composite image is displayed again on confirmation screen page d3 as shown in FIG. 9 (b) (step S7).

When a technician confirms breast images on the confirmation screen page d3, and judges that the correspondence relationship between the radiographing order information and breast images is correct from the position of a stamp image, the technician presses output button k2. On the other hand, when further correction is needed, correction button k1 is pressed. On the control device 30, when correction button k1 is pressed, the process returns to process of step S4, and processes S5-S7 are repeated. On the other hand, if output button k2 is pressed (step S8; YES), the corrected radiographing order information is caused to accompany the breast image, and the breast image is stored in image memory 36 (step S9). Then, data of composite image displayed on confirmation screen page d3 are outputted toward film outputting device 70, and the present processing terminates. After that, film output of composite image is conducted on the film outputting device 70.

Since the stamp image is arranged at the position where the correct and the incorrect of the correspondence relationship between breast images and radiographing order information can be distinguished, in the composite image of each breast image as stated above, a technician can find an error of the correspondence relationship easily from the positional relationship between a breast area and a stamp image, and wrong image control can be prevented.

Further, since the registration information of cassette ID for making radiographing order information and breast images to correspond to each other is made to be of the correctable structure, the correspondence relationship can be corrected easily, when the correspondence relationship is determined to be wrong, and the accuracy of image control can be maintained.

Incidentally, the embodiment stated above is a preferred example to which the invention is applied. However, the invention is not limited to this. For example, though the stamp images are superposed after arranging breast images for left and right breasts in the aforesaid explanation, it is also possible to conduct rotation and arrangement of breast images after superposing stamp images on each breast image first. In this case, it is necessary to rotate characters of stamp images in advance, so that the orientation of characters of stamp images may become the orientation readable by a technician on the composite image after arrangement. With respect to the position where a stamp image is arranged, it is preferable to establish it in advance corresponding to the radiographing region and the radiographing direction so that the stamp image is arranged on a hollow area when the breast image is corresponding correctly to the radiographing order information, and a stamp image may be arranged at the position where the stamp image is overlapped with a breast image when the breast image is corresponding incorrectly to the radiographing order information, such as lower on the right in MLO-L, and upper on the right in MLO-R when a chest wall side is on the right side.

Further, the stamp image may also be an icon that means nothing, without being limited to characters showing radiographing performance conditions and radiographing order information. In other words, everything can be used, provided that a technician can observe the image visually on the display screed page.

In the embodiment stated above, breast images only for MLO have been described. On the occasion where four breast images in total are acquired in the radiographing direction of CC in addition to MLO, if the correspondence relationship between the radiographing order information and breast images is wrong for breast image for MLO, there is a possibility that the correspondence relationship between the radiographing order information and breast images is wrong also for breast images for CC.

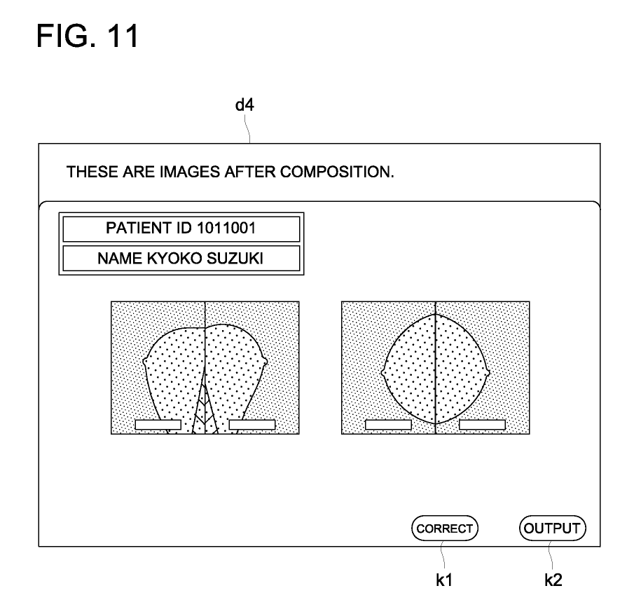

For example, when radiographing is conducted in the order of MLO-L, MLO-R, CC-L and CC-R despite cassette registration for four cassettes conducted in the order of MLO-R, MLO-L, CC-R and CC-L, breast images for MLO and CC are caused by the cassette ID to correspond to radiographing order information for breast regions which are left-right reversal respectively. Therefore, if composite images are prepared from breast images under the condition of one set including two sheets of breast images in respective radiographing directions, composite images shown in FIG. 11 are displayed on confirmation screen page d4. As shown in FIG. 11, breast images and stamp images are overlapped for breast images in MLO, because breast regions are rotated under the left-right reversal state. In this case, breast images in CC look to have no problem at first sight. However, breast regions are arranged on a left-right reversal basis, and if the stamp image compounded is based on radiographing order information, contents thereof are also wrong.

Even in this case, it is easy to correct the correspondence relationship between radiographing order information and breast images, under the assumption that left and right breasts are radiographed by turns at the time of radiographing, and reading of a breast image from cassette c is conducted for each radiographing. In the case of radiographing, it is common that left and right breasts are radiographed by turns to avoid pains of a patient caused by continuous compression on the same breast.

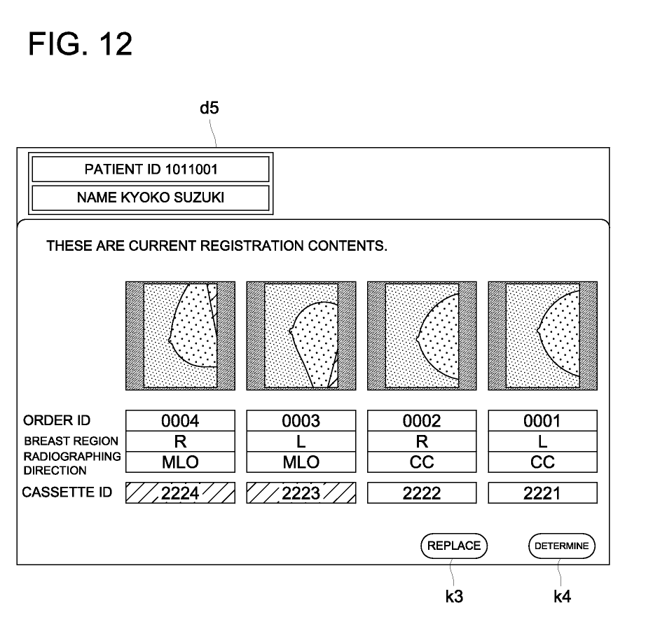

When correction button k1 is pressed down on confirmation screen page d4, correction screen page d5 shown in FIG. 12 is displayed, and respective breast images are displayed in the order in which they are acquired. Since the breast image is read for each radiographing in this case, the order of acquisition agrees with the order of radiographing. On this correction screen page d5, a technician can identify the breast image in MLO visually based on a position and a form of a breast area, and also can identify left and right of breast regions based on a position of a pectoral muscle section. Therefore, the technician identifies left and right of breast regions for breast images in MLO first, and if the technician judges, from contents of present radiographing order information, that the radiographing order information correlated with two breast images is opposite, the technician presses down replacement button k3 after selecting cassette ID of each breast image in MLO. On the control device 30, cassette ID selected in order file 351 is replaced to be stored updated, and is corrected. Order ID and contents of the order of the radiographing order information after the correction are reflected on the correction screen page d5.

Then, the technician can distinguish left and right of breast regions in breast images in CC depending on whether the breast image in MLO after correction is for the left or right breast region or depending on the order of acquisition of breast images. In other words, the order of arrangement of breast images in CC which are arranged next to breast images in MLO arranged in the order of L→R should also be L→R because breasts are radiographed alternately for left and right in the case of radiographing. Owing to this, the correct and the incorrect of the correspondence relationship between radiographing order information and breast images can be discriminated also for breast images in CC, thus, replacement button k3 is pressed down after selecting cassette ID for replacement, in the same way as in the occasion of MLO. In the control device 30, selected cassette ID is replaced to be updated and stored in order file 351, and the correspondence relationship between breast images and radiographing order information is corrected.

Though an example of using a cassette as an X-ray detector was described in the aforesaid embodiment, it is also possible to apply the invention even when FPD (Flat Panel Detector) is used, without being limited to the foregoing. In the case of FPD, radiographing is repeated by using one sheet of FPD usually, and data of breast images are generated for each radiographing to be transmitted to the control device 30. When a technician instructs the order of radiographing for plural radiographing order information for a subject of one person (identification information in the present invention), correlation of acquired breast images is conducted based on the order of radiographing instructed for plural radiographing order information, on the control device 30. In this case, even when radiographing is carried out in an order that is different from the order of radiographing instructed by the technician by mistake, the technician can notice an error of the correspondence relationship easily, by preparing a composite image and displaying a stamp image on an overlapping basis, as in the aforesaid embodiment. Then, when an error of the correspondence relationship is found out, it is possible to correct the error by replacing the order of radiographing for the radiographing order information.

Incidentally, it is also possible to constitute so that data of breast images generated for each radiographing are preserved in a memory housed in FPD in sequence. In this case, if data of breast images equivalent to one patient preserved in a memory (for example, data of breast images for four times of radiographing) are transmitted in sequence to the control device 30, beginning with data of breast images taken first, after completion of all operations of radiographing for one patient, breast images can be made to correspond based on the order of radiographing instructed for radiographing order information, on the control device 30.

1. A breast image generating method, comprising the steps of:
correlating each of a plurality of pieces of radiographing order information for instructing to radiograph each of left and right breasts of a single subject in an oblique direction with identification information indicating a relationship between data of a breast image and the each of the plurality of pieces of radiographing order information so as to be stored in a storing device;
radiographing a breast based on the each of the plurality of pieces of radiographing order information to generate data of a breast image;
identifying each of the plurality of pieces of radiographing order information corresponding to the generated data of the breast image based on the identification information; and
superposing a stamp image at a prescribed position on the breast image taken through radiographing conducted in the oblique direction to make a composite image in accordance with whether a breast region indicated by the identified each of the plurality of pieces of radiographing order information is left or right, after the plurality of pieces of radiographing order information for data of breast images for a single subject are identified, so as to display the composite image on a displaying device.

2. A breast image generating method, comprising the steps of:
correlating each of a plurality of pieces of radiographing order information for instructing to radiograph each of left and right breasts of a single subject in an oblique direction and a vertical direction with identification information indicating a relationship between data of a breast image and the each of the plurality of pieces of radiographing order information so as to be stored in a storing device;
radiographing a breast based on the each of the plurality of pieces of radiographing order information to generate data of a breast image;
identifying each of the plurality of pieces of radiographing order information corresponding to the data of the breast image based on the identification information; and
superposing a stamp image at a prescribed position on the breast image taken through radiographing conducted in the oblique direction to make a composite image in accordance with whether a breast region indicated by the identified each of the plurality of pieces of radiographing order information is left or right, after the plurality of pieces of radiographing order information for data of breast images for a single subject are identified, so as to display the composite image on a displaying device.

3. The breast image generating method according to claim 1,
wherein, when correction of the identification information is instructed through an operation device after the composite image in which the stamp image is superposed is displayed, respective breast images for a single subject are arranged to be displayed in an order in which the breast images have been generated.

4. The breast image generating method according to claim 3, further comprising the step of:
correcting identification information correlated with the each of the plurality of radiographing order information, in accordance with an instruction of correction for a correspondence relationship between the breast image and the each of the plurality of radiographing order information, the instruction being inputted through an operation device, after displaying the respective breast images in an order in which the breast images are generated, so as store the corrected identification information in the storing device.

5. The breast image generating method according to claim 4, further comprising the steps of:
identifying each of the plurality of radiographing order information corresponding to each of one set of left and right breast images taken through radiographing in the oblique direction, based on the corrected identification information, and
superposing a stamp image at a prescribed position on the breast image taken through radiographing in the oblique direction to make a composite image depending on whether a breast region indicated by the identified each of the plurality of radiographing order information is left or right so as to display the composite image again on a displaying device.

6. The breast image generating method according to claim 2,
wherein, when correction of the identification information is instructed through an operation device after the composite image in which the stamp image is superposed is displayed, respective breast images for a single subject are arranged to be displayed in an order in which the breast images have been generated.

7. The breast image generating method according to claim 6, further comprising the step of:
correcting identification information correlated with the each of the plurality of radiographing order information, in accordance with an instruction of correction for a correspondence relationship between the breast image and the each of the plurality of radiographing order information, the instruction being inputted through an operation device, after displaying the respective breast images in an order in which the breast images are generated, so as store the corrected identification information in the storing device.

8. The breast image generating method according to claim 7, further comprising the steps of:
identifying each of the plurality of radiographing order information corresponding to each of one set of left and right breast images taken through radiographing in the oblique direction, based on the corrected identification information, and
superposing a stamp image at a prescribed position on the breast image taken through radiographing in the oblique direction to make a composite image depending on whether a breast region indicated by the identified each of the plurality of radiographing order information is left or right so as to display the composite image again on a displaying device.
